(19) United States
(12) Patent Application Publication
(10) Pub. No.: US 2003/0013835 A1
(43) Pub. Date: Jan. 16, 2003

(54) 100% polyurea elastomeric system, process for producing and use thereof

(76) Inventors: Anastasios Zahariadis, Santa Ana, CA (US); Gerald Thermos, Seal Beach, CA (US)

Correspondence Address: THOMPSON COBURN, LLP, ONE FIRSTAR PLAZA, SUITE 3500, ST LOUIS, MO, 63101, US

(21) Appl. No.: 09/843,332

(22) Filed: Apr. 25, 2001

(51) Int. Cl. C07C 273/00

(52) U.S. Cl. 528/44; 528/60; 528/61; 528/64; 528/68; 564/32; 564/47; 564/48; 564/49; 564/53

(57) ABSTRACT

A polyurea composition free from polyol linkages. A composition and process for producing a two component spray 100% polyurea elastomeric system wherein an A and a B component are mixed, wherein said A component is a prepolymer produced by an aromatic isocyanate and amine terminated materials and wherein said B component is 100% amine terminated reactive ingredients. The instant invention can be used as a spray system for lining and coating surfaces.

### BACKGROUND OF THE INVENTION

Polyurea is a relatively new polymer on the market which has many advantages over existing polymer systems and coatings. Polyurea is a high performance sprayed plural component elastomer which provides an extremely tough, monolithic membrane with excellent water and good chemical resistance.

Polyurea bonds have the chemical structure given below.

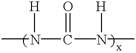

Polyurea has an extremely high resistance to abrasion and, unlike polyurethanes and epoxies, is hydrophobic. These characteristics are an improvement over polyurethanes and epoxies in that polyurea is affected very little by damp or cold surfaces. Polyurea can also be sprayed directly on water or ice or at very cold temperatures (even below freezing) with minimal effect on tack free time. Because polyurea is impervious to standing water, it is an excellent choice for lining concrete and steel tanks, ponds, troughs and many types of roofing. Further, polyurea chemistry is quite resistant to a broad range of corrosives.

Polyurea has a number of potential applications, such as protecting secondary containments and exposed structures, such as bridges, steel tanks, piping, metal buildings, and practically any surface where corrosion exists or can be a problem. Polyurea has also been widely utilized as a coating for roofs, walls, and floors as well as for protecting steel and wood pilings.

Polyureas currently available on the market are not true polyureas in that they contain some polyol linkages and most contain as little as 50% aromatic polyurea. Polyol linkages are not desirable in all applications because these linkages open up the polyurea to degradation in certain environments.

The instant invention overcomes these problems associated with current polyureas.

### OBJECTS OF THE INVENTION

It is an object of this invention to provide a 100% aromatic polyurea free from polyols as well as a process for producing and process of using the same.

It is a further object of this invention to provide a composition and process for producing a two component spray 100% polyurea elastomeric system comprising an A and a B component wherein said A component comprises a prepolymer comprising an aromatic isocyanate as a hard segment and an amine terminated material (preferably an amine, diamine or triamine, most preferably a diamine or triamine) as a soft segment (chain extender) and wherein said B component comprises 100% amine terminated reactive ingredients.

These and other objects are provided and described in more detail hereafter.

### BRIEF SUMMARY OF THE INVENTION

This invention comprises a unique two component spray 100% polyurea elastomeric system. The 100% polyurea elastomeric system of the instant invention is produced by mixing an A Component and a B Component. The A Component of the instant invention comprises a manufactured prepolymer comprising an aromatic isocyanate and an amine terminated material, preferably an amine, diamine or triamine, most preferably a diamine or triamine, as a chain extender. The B Component of the instant invention comprises 100% amine terminated reactive ingredients. The mixture of the A and B component of the instant invention produces a 100% polyurea elastomeric system free from polyol linkages. This system can then be used as a spray system for lining and coating surfaces.

### DETAILED DESCRIPTION OF THE INVENTION

Polyureas are known which contain polyol linkages. A polyurea free of polyol linkages however has advantages over polyurea with polyol linkages including increased stability, increased tensile strength and increased tear strength as well as improvements in chemical resistance and hydrolytic stability. The instant invention is a polyurea free of polyol linkages.

The instant invention comprises a two component spray 100% polyurea elastomeric system comprising an A and a B component wherein said A component comprises a prepolymer produced from an aromatic isocyanate as a hard segment and an amine terminated material (preferably an amine, diamine or triamine, most preferably a diamine or triamine) as a soft segment (chain extender) and wherein said B component comprises 100% amine terminated reactive ingredients. The composition of the instant invention is essentially free from polyol bonds and in the preferred embodiment is free from polyol linkages. The process for producing the 100% polyurea of the instant invention produces a polyurea free from polyol linkages.

The A Component of the instant invention comprises a manufactured prepolymer produced from isocyanates, amine terminated materials or combinations thereof. Preferred amine terminated materials useful to produce the A Component include amines, diamines and triamines with diamines and triamines as the most preferred amine terminated materials.

Isocyanates useful to produce the prepolymer of the instant invention have the general formula

OCN—R—NCO

below wherein R comprises aromatic groups.

Nonlimiting examples of isocyanates useful to produce the prepolymer of the instant invention include isocyanates of the formulas

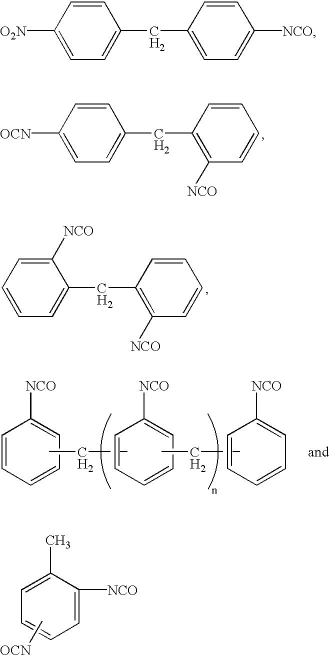

and are available from Bayer Corporation (100 Bayer Road, Pittsburgh, Pa. 15205-9741), The Dow Chemical Company (2030 Dow Center Drive, Midland, Mich. 48642) and ICI/Huntsman Corporation (500 Huntsman Way, Salt Lake City, Utah 84108).

Diamines or triamines useful to produce the prepolymer of Component A of the instant invention comprise diamines and triamines of the general formulas

H₂N—R₁—NH₂

or

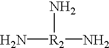

wherein R₁ and R₂ comprise polyoxyalkylene groups. Polyoxyalkylenes useful in the instant invention are available from the JEFFAMINE® line of products (Huntsman Corporation, 500 Huntsman Way, Salt Lake City, Utah 64108).

Nonlimiting examples of polyoxyalkylenes useful as R₁ of the instant invention include but are not limited to polyoxyalkylenes of the general formulas

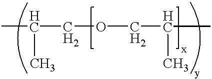

wherein x is 2-68 (more preferably x=2-3, 5-6, 33 and 68), and y is any whole number;

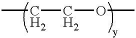

wherein y is any whole number;

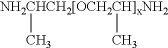

wherein x is 25-40 (most preferably about 33);

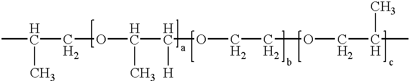

wherein a+c=5 and b=38-39; and

NH₂CH₂CH₂OCH₂CH₂OCH₂CH₂NH₂

Nonlimiting examples of polyoxyalkylenes useful as R₂ in the triamines of Component A of the instant invention include but are not limited to those of the general formulas

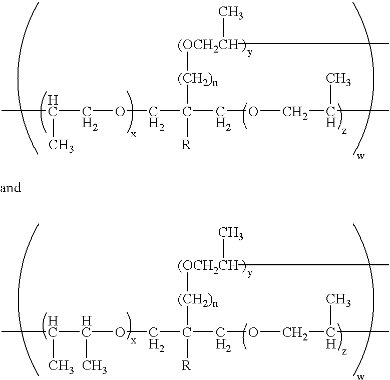

wherein x+y+z=5-81, n is a whole number, and w is a whole number (preferred embodiments of x+y+z=5-6, 50 and 81).

The prepolymer produced from the mixture of the isocyanates, diamines and triamines of the instant invention has the general formula

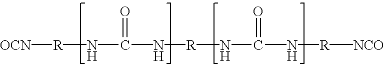

wherein R comprises aromatic groups or polyoxyalkylene units such as the aromatic groups or polyoxyalkylenes earlier defined in this specification as suitable for the practice of the instant invention. Polyurea bonds are indicated in brackets. As can be seen, the prepolymer is produced free of polyol bonds.

The B Component of the instant invention can be produced from a first component comprising diamines, triamines or mixtures thereof of the general formula

H₂N—R₁—NH₂

or

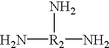

and a second component comprising a diamine of the general formula

H₂N —Ar—NH₂

wherein Ar represents an aromatic group. Nonlimiting examples of diamines of the general formula useful in the practice of the instant invention include but are not limited compounds of the formulas

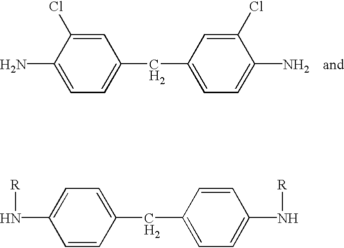

and are available from Albemarle Corporation (451 Florida Street, Baton Rouge, La. 70801-1765) and Bayer Corporation (100 Bayer Road, Pittsburgh, Pa. 15205-9741).

Diamines further useful in the B Component of the instant invention comprise those of the general formula

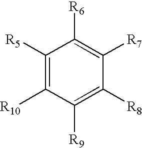

wherein R₆-R₁₀ are selected from the group consisting of H, CH₃, CH₂CH₃, NH₂ or SCH₃ wherein at least two groups of R₆-R₁₀comprise NH₂ groups. Diamines of the specific formulas

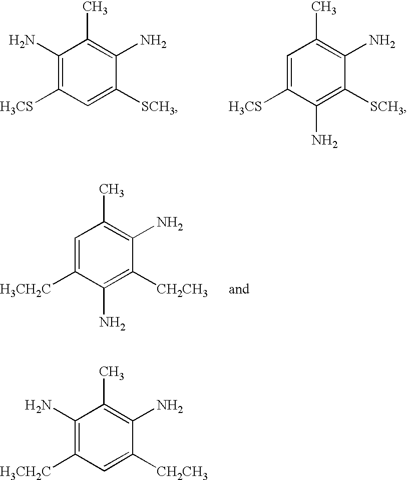

are further nonlimiting examples of diamines useful as the B component of the instant invention.

The process of producing the 100% polyurea elastomeric system of the instant invention comprises the mixing of the prepolymer of Component A produced above with the Component B. The mixture of Component A with Component B of the instant invention produces the 100% polyurea elastomeric system and can be depicted by the general equation

OCN—R—NCO+H₂N—R₁—NH₂+H₂N—Ar—NH₂→Urea Bonded Elastomer

and/or

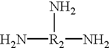

wherein R, R₁, R₂ and Ar are as earlier defined in this specification.

The weight percentage, mole percentage or volume of starting components will vary depending upon the equipment utilized, the starting components activities and the desired product's characteristics. General formulations envisioned of Component A and Component B useful to produce the 100% polyurea elastomeric system of the instant invention comprise from about 20-80% Component A to about 80-20% component B, more preferably from about 40-60% Component A to about 60-40% component B, and most preferably a 50%-50% mixture by volume of Component A and Component B.

The produced urea bonded elastomer of the instant invention has the general formula indicated above.

### Producing the Prepolymer

The manufacturing of the prepolymer of Component A comprises the steps of placing an aromatic isocyanate and a diluent into a reactor vessel. Nonlimiting examples of diluents useful in the practice of this invention include but are not limited to propylene carbonate, propylene oxide and propylethylene oxide. It may be necessary in some instances to add an organic acid to the reactor vessel also in order to adjust the pH, increase the affinity of the isocyanate and decreases the reactivity of the amine so as to optimize reaction conditions. This mixture is then blended in a blending tank, reaction vessel or other similar instrumentality while maintaining the temperature in a range from about 60-300° F. although a more preferable temperature range is from about 120-200° F. The mixture can be heated by an external heat source or the heat can be generated by exotherms produced by the reaction of the isocyanate and amine itself, depending on which starting components are utilized.

Using a high RPM sheer mixer the amine terminated material (amine, diamine or triamine) is added in small increments while maintaining the temperature at all times during the manufacturing procedure. The size of the increments of amine terminated material to be added will depend on the profile of the activity for the reaction of the isocyanate and amine. The increments are envisioned to be of any small size, and should not exceed 10% of the amount of isocyanate in the mixture.

The material produced should then be titrated after 24 hours to verify the desired NCO content.

### Producing the 100% Polyurea Elastomeric System of the Instant Invention

The prepolymer of the instant invention produced by the above process can then be mixed with 20-80%, 40-60% and most preferably a 50% weight percentage or percentage by volume of component B in order to produce the 100% polyurea two component spray of the instant invention.

### Using the 100% Polyurea Elastomeric System of the Instant Invention

The polyurea of the instant invention is useful for spraying, rolling, caulking or trowelable type uses. Spraying of the instant invention would be accomplished by a high or low pressure spray gun or similar instrumentalities. Rolling of the instant invention would be accomplished by any suitable roller such as an equipment roller or a manual roller. Caulking of the instant invention could be accomplished by utilizing caulking guns, caulking machines or the like. A trowel is envisioned when utilizing the instant invention in trowelable type uses.

Polyureas of the instant invention are useful as coatings, joint fillers, in erosion prevention, abrasion prevention, encapsulation and other similar processes. Specific uses wherein the instant invention has advantages over commercially available polyureas would be in the protection of anaerobic digestion tank liners.

The produced 100% polyurea elastomeric system of the instant invention has the advantage of being produced essentially free of polyol linkages, with the preferred embodiment of the instant invention free from polyol linkages.

### EXAMPLES

The examples herein are illustrations of various embodiments of this invention and are not intended to limit it in any way.

### EXAMPLE I

### Preparation of the Prepolymer of the Instant Invention

The manufacturing of the prepolymer consists of the following processing steps: Mondur ML, an aromatic isocyanate from Bayer Corporation (100 Bayer Road, Pittsburgh, Pa. 15205-9741), benzoyl chloride (an organic acid), and propylene carbonate (a diluent) were placed into a reactor vessel. The reaction vessel's contents were then blended and heated at about 160° F., while maintaining constant temperature.

A high RPM sheer mixer was then utilized while Jeffamine D-2000 (a diamine available from Huntsman Corporation (500 Huntsman Way, Salt Lake City, Utah 84108)) was added in small increments not exceeding 10% of the concentration of isocyanate while maintaining a temperature of about 160° F. at all times. A high RPM was maintained during the manufacturing procedure.

The material produced was then titrated by 0.1 M HCl after 24 hours to verify the desired NCO content. In this instance, the desired NCO content was approximately 15% NCO.

### Example II

### Titration

The percent weight of free NCO was determined by weighing 0.2-0.3 grams of the material produced into a clean, dry 250 ml Erlenmeyer Flask. 100 cc of toluene was added to the flask to ensure the sample completely dissolved (using heat if necessary). 25 ml of dibutyl amine-toluene solution was then added to the flask. The mixture was then allowed to stand for 15-20 minutes. 100 ml of isopropyl alcohol was then added to the flask with heat to ensure that everything was dissolved in the alcohol (being careful not to bring the mixture to a boil). 4-5 drops of bromophenol blue indicator was then added. The entire solution was then titrated with 0.1 N HCl solution until the color of the liquid in the flask changed from blue to yellow.

The percent of NCO was calculated using the formula below:

$$\% NCO = \frac{\text{(Blank-Titration)} \times 4.2 \times N}{\text{Sample Weight}}$$

wherein N stands for the normality of the hydrochloric acid solution (approximately 0.1). The final NCO content was determined to be 15%.

### Example III

### Embodiment of 100% Polyurea Formulation

The following is an example of a 100% polyurea formulation. Percentages will vary depending on the type of aromatic isocyanate utilized and upon the desired ratio of components.

| Component A: | |
|---|---|
| Aromatic Isocyanate | 45% |
| Benzoyl Chloride | 80 grams/2000 lbs |
| Propylene Carbonate | 10% |
| Polyoxypropylenediamine | 45% |
| Component B: | |
| Polyoxypropylenediamine | 71.4% |
| Diethytoluenediamine (DETA) | 26.1% |
| Pigment | 1% |
| UV inhibitor/antioxidants | 1.5% |

Thus, it is apparent that there has been provided, in accordance with the instant invention, a process that fully satisfies the objects and advantages set forth herein above. While the invention has been described with respect to various specific examples and embodiments thereof, it is understood that the invention is not limited thereto and many alternatives, modifications and variations will be apparent to those skilled in the art in light of the foregoing description. Accordingly, it is intended to embrace all such alternatives, modifications and variations as fall within the spirit and broad scope of the invention.

What is claimed is:

1. A composition comprising a 100% aromatic polyurea essentially free of polyols.

2. The composition of claim 1 wherein said polyurea is free of polyols.

3. A 100% aromatic polyurea comprising an A Component and a B Component,
a) said A Component comprising a manufactured prepolymer comprising an aromatic isocyanate and an amine terminated material as a chain extender and
b) said B component comprising 100% amine terminated reactive ingredients, wherein said polyurea is essentially free of polyols.

4. The 100% aromatic polyurea of claim 3 further comprising a polyurea free of polyols.

5. The 100% aromatic polyurea of claim 4 comprising where said isocyanate comprises the general formula
OCN—R—NCO
wherein R comprises an aromatic group.

6. The 100% aromatic polyurea of claim 5 wherein said isocyanate is selected from the group consisting of

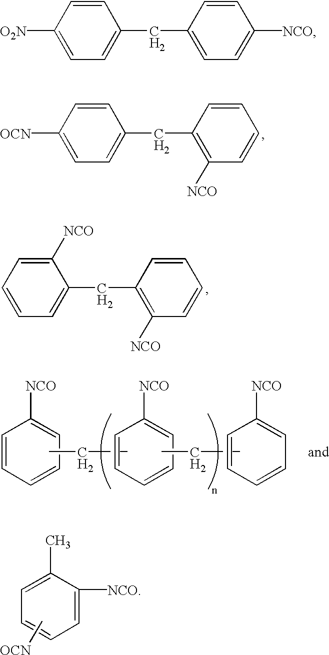

7. The 100% aromatic polyurea of claim 5 wherein said amine terminated material of component A is selected from the group comprising amines, diamines or triamines.

8. The 100% aromatic polyurea of claim 7 wherein said amine terminated material of component A is selected from the group consisting of diamines or triamines of the general formula
H₂N—R₁—NH₂
or 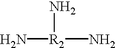
wherein R₁ and R₂ comprise polyoxyalkylenes.

9. The 100% aromatic polyurea of claim 8 wherein said polyoxyalkylenes are selected from the group consisting of polyoxyalkylenes of the general formulas 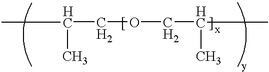
wherein x is 2-68 and y is any whole number; 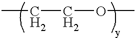
wherein y is any whole number; 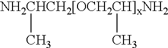
wherein x is 25-40 (most preferably about 33); 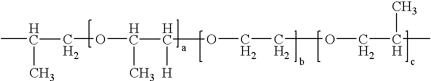
wherein a+c=5 and b=38-39; 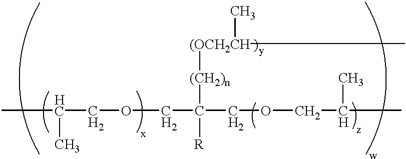
wherein x+y+z=5-81, n=a whole number and w=a whole number; 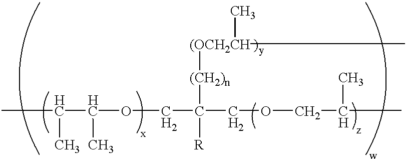
wherein x+y+z=5-81, n=a whole number and w=a whole number; and
NH₂CH₂CH₂OCH₂CH₂OCH₂CH₂NH₂

10. The 100% aromatic polyurea of claim 5 wherein said prepolymer comprises the general formula 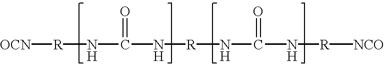
wherein R is an aromatic group.

11. The 100% aromatic polyurea of claim 10 wherein said prepolymer is free from polyol linkages.

12. The 100% aromatic polyurea of claim 5 wherein said second component comprises diamines of the general formula
H₂N—Ar—NH₂
wherein Ar comprises aromatic groups.

13. The 100% aromatic polyurea of claim 5 wherein said second component is selected from the group consisting of diamines of the formulas:

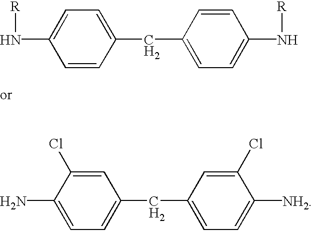

14. The 100% aromatic polyurea of claim 12 wherein said second component is selected from the group consisting of diamines of the general formula: 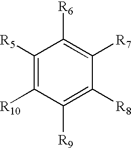
wherein R₆-R₁₀ are selected from the group consisting of H, CH₃, CH₂CH₃, NH₂ or SCH₃ wherein at least two groups of R₆-R₁₀ comprise NH₂ groups.

15. The 100% aromatic polyurea of claim 14 wherein said diamine is selected the group consisting of

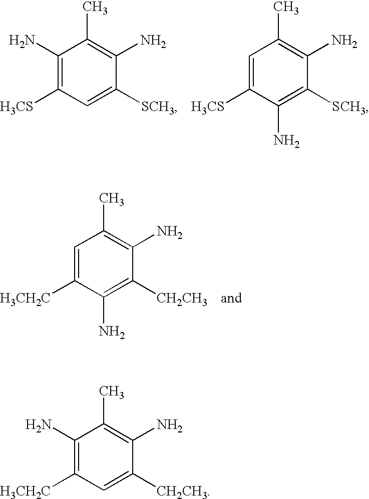

16. A process of preparing a prepolymer for use in producing a 100% aromatic polyurea comprising:
a) the addition of an aromatic isocyanate and a diluent into a reactor vessel to produce a reaction solution,
b) mixing said reaction solution
c) adding small increments of diamine to said reaction solution wherein said diamine and reaction solution are mixed utilizing a high RPM sheer mixer,
d) titrating the product after 24 hours to verify NCO content
wherein said prepolymer is essentially free of polyols.

17. The process of claim 16 comprising the addition of an organic acid into the reactor vessel with the aromatic isocyanate and diluent to produce a reaction solution.

18. The process of claim 17 wherein said reaction solution is mixed at a temperature of about 60-300° F.

19. The process of claim 18 wherein said reaction solution is mixed at a temperature of about 20-200° F.

20. The process of claim 18 wherein said diamine and reaction solution are mixed while maintaining temperature.

21. The process of claim 16 further comprising the addition of the prepolymer to a B component wherein said B component comprises 100% amine terminated reactive ingredients to produce a 100% aromatic polyurea.

22. The process of claim 16 wherein said B Component comprises a first component comprising diamines, triamines or mixtures thereof and a second component comprising diamines.

23. The process of claim 22 wherein said isocyanate further comprises the general formula
OCN—R—NCO
wherein R comprises an aromatic group.

24. The process of claim 22 wherein said first component comprises diamines or triamines of the formulas
H₂N—R₁—NH₂
or

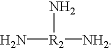

25. The process of claim 24 wherein the R₁ group of said diamines consists of polyoxyalkylene groups of the general formulas 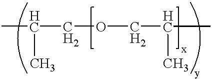
wherein x is 2-68 and y is any whole number; 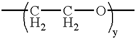
wherein y is any whole number; 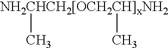
wherein x is 25-40 (most preferably about 33); 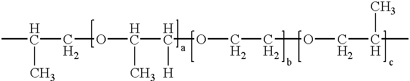
wherein a+c=5 and b=38-39; and
NH₂CH₂CH₂OCH₂CH₂OCH₂CH₂NH₂

26. The process of claim 24 wherein the R₂ group of said triamines consists of polyoxyalkylene groups of the general formula 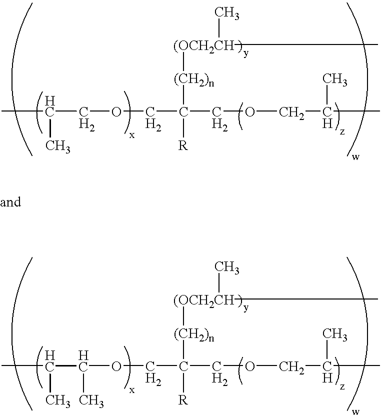
wherein x+y+z=5-81, n=a whole number and w=a whole number.

27. The process of claim 22 wherein said second component comprises diamines of the general formula
H₂N—Ar—NH₂
wherein Ar comprises aromatic groups.

28. The process of claim 27 wherein said second component is selected from the group consisting of diamines of the formulas

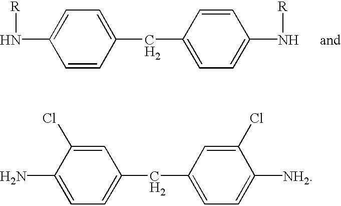

29. The process of claim 27 wherein said second component is selected from the group consisting of diamines of the general formula: 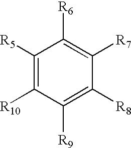
wherein R₆-R₁₀ are selected from the group consisting of H, CH₃, CH₂CH₃, NH₂ or SCH₃ wherein at least two groups of R₆-R₁₀ comprise NH₂ groups.

30. The process of claim 29 wherein said second component is selected from the group consisting of

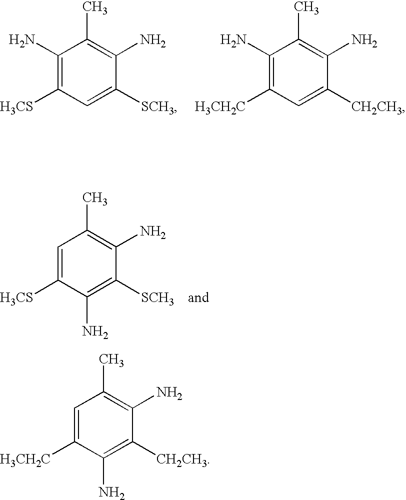

31. A process of using a two component spray 100% polyurea elastomeric system free from polyol linkages comprising lining a surface by spraying, caulking, rolling-on or trowelling said two component spray system onto a surface.

32. The process of claim 31 wherein said surface comprises concrete, steel, wood, pond beds, troughs, roofs, containments, secondary containments, exposed structures, bridges, steel tanks, pipes, metal buildings and the like.
